(19) United States
(12) Patent Application Publication
(10) Pub. No.: US 2026/0102087 A1
(43) Pub. Date: Apr. 16, 2026

(54) SIGNAL PROCESSING FOR MONOCHROMATIC DETECTORS

(71) Applicant: Cardiac Pacemakers, Inc., St Paul, MN (US)

(72) Inventors: Michael J. Kane, St. Paul, MN (US); Yingbo Li, Woodbury, MN (US)

(21) Appl. No.: 19/356,353

(22) Filed: Oct. 13, 2025

Related U.S. Application Data
(60) Provisional application No. 63/706,886, filed on Oct. 14, 2024.

(51) Int. Cl. A61B 5/1459

(52) U.S. Cl. CPC A61B 5/1459; A61B 2562/0233

(57) ABSTRACT

An apparatus includes an opto-electronic assembly and circuitry. The opto-electronic assembly includes a substrate, a first optical emitter coupled to the substrate and configured to emit light at a first wavelength, and a second optical emitter coupled to the substrate and configured to emit light at a second wavelength different than the first wavelength. The circuitry is configured to: apply a first current level to the first optical emitter and apply a second current level to the second optical emitter. The second current level is greater than the first current level.

### CROSS REFERENCE TO RELATED APPLICATION

This application claims the benefit of Provisional Application No. 63/706,886, filed October 14, 2024, which is incorporated herein by reference in its entirety for all purposes.

### TECHNICAL FIELD

Instances of the present disclosure relate to electronic and/or optical components for use with chemical sensors.

### BACKGROUND

Chemical sensors can be used to measure patients’ physiological parameters.

### SUMMARY

In Example 1, an apparatus includes an opto-electronic assembly and circuitry. The opto-electronic assembly includes a substrate (e.g., a circuit board), a first optical emitter coupled to the substrate and configured to emit light at a first wavelength, and a second optical emitter coupled to the substrate and configured to emit light at a second wavelength different than the first wavelength. The circuitry is configured to: apply a first current level to the first optical emitter, and apply a second current level to the second optical emitter, wherein the second current level is greater than the first current level.

In Example 2, the apparatus of Example 1, wherein the circuitry is further configured to: apply the first current level to the first optical emitter such that light emission from the first optical emitter is pulsed, and apply the second current level to the second optical emitter such that light emission from the second optical emitter is pulsed.

In Example 3, the apparatus of Examples 1 or 2, wherein the circuitry is further configured to: apply the first current level to the first optical emitter such that light emission from the first optical emitter is pulsed a first set number of times before the second current level is applied to the second optical emitter such that light emission from the first optical emitter is pulsed a second set number of times.

In Example 4, the apparatus of Example 3, wherein the first set number is 5–100, wherein the second set number is 5–100.

In Example 5, the apparatus of Examples 2–4, wherein each pulse lasts 5–120 microseconds.

In Example 6, the apparatus of Examples 1–5, wherein the opto-electronic assembly further includes an optical detector coupled to the substrate.

In Example 7, the apparatus of Example 6, wherein the circuitry is further configured to adjust a gain of the optical detector in response to whether the first optical emitter or the second optical emitter is being pulsed.

In Example 8, the apparatus of Examples 6 or 7, wherein the circuitry is further configured to apply a signal averaging technique to reduce noise in a signal generated by the optical detector.

In Example 9, the apparatus of any of Examples 1–8, wherein the apparatus is a medical device, wherein the medical device includes a chemical indicator arranged to reflect light from the first optical emitter and the second optical emitter, wherein the medical device is programmed to estimate an analyte concentration based, at least in part, on an optical property of the chemical indicator.

In Example 10, the apparatus of Example 9, wherein the analyte concentration estimated is further based, at least in part, on an estimated temperature of the chemical indicator.

In Example 11, the apparatus of Example 10, wherein the estimated temperature is generated by a temperature sensor coupled to the substrate or an optical detector.

In Example 12, the apparatus of any of Examples 9–11, wherein the analyte concentration estimated is further based, at least in part, on an estimated pressure.

In Example 13, the apparatus of any of Examples 9–12, wherein the analyte concentration estimated is further based, at least in part, on a sleep state or active state of a patient.

In Example 14, the apparatus of any of Examples 1–13, wherein the first wavelength is 510–570 nm, wherein the second wavelength is 610–740 nm.

In Example 15, the apparatus of any of Examples 1–14, wherein the first optical emitter and the second optical emitter are light emitting diodes.

In Example 16, an apparatus includes an opto-electronic assembly and circuitry. The opto-electronic assembly includes a substrate, a first optical emitter coupled to the substrate and configured to emit light at a first wavelength, and a second optical emitter coupled to the substrate and configured to emit light at a second wavelength different than the first wavelength. The circuitry is configured to: apply a first current level to the first optical emitter, and apply a second current level to the second optical emitter, wherein the second current level is greater than the first current level.

In Example 17, the apparatus of Example 16, wherein the circuitry is further configured to: apply the first current level to the first optical emitter such that light emission from the first optical emitter is pulsed, and apply the second current level to the second optical emitter such that light emission from the second optical emitter is pulsed.

In Example 18, the apparatus of Example 16, wherein the circuitry is further configured to: apply the first current level to the first optical emitter such that light emission from the first optical emitter is pulsed a first set number of times before the second current level is applied to the second optical emitter such that light emission from the first optical emitter is pulsed a second set number of times.

In Example 19, the apparatus of Example 18, wherein the first set number is 5–100, wherein the second set number is 5–100.

In Example 20, the apparatus of Example 19, wherein each pulse lasts 5–120 microseconds.

In Example 21, the apparatus of Example 16, wherein the opto-electronic assembly further includes an optical detector coupled to the substrate.

In Example 22, the apparatus of Example 21, wherein the circuitry is further configured to adjust a gain of the optical detector in response to whether the first optical emitter or the second optical emitter is being pulsed.

In Example 23, the apparatus of Examples 21, wherein the circuitry is further configured to apply a signal averaging technique to reduce noise in a signal generated by the optical detector.

In Example 24, the apparatus of Example 16, wherein the apparatus is a medical device, wherein the medical device includes a chemical indicator arranged to reflect light from the first optical emitter and the second optical emitter, wherein the medical device is programmed to estimate an analyte concentration based, at least in part, on an optical property of the chemical indicator.

In Example 25, the apparatus of Example 24, wherein the analyte concentration estimated is further based, at least in part, on an estimated temperature of the chemical indicator.

In Example 26, the apparatus of Example 25, wherein the estimated temperature is generated by a temperature sensor coupled to the substrate or an optical detector.

In Example 27, the apparatus of Example 24, wherein the analyte concentration estimated is further based, at least in part, on an estimated pressure.

In Example 28, the apparatus of Example 24, wherein the analyte concentration estimated is further based, at least in part, on a sleep state or active state of a patient.

In Example 29, the apparatus of Example 16, wherein the first wavelength is 510–570 nm, wherein the second wavelength is 610–740 nm.

In Example 30, a method is disclosed for use with an implantable medical device with a substrate, a first optical emitter coupled to the substrate, and a second optical emitter coupled to the substrate. The method includes applying a first current level to the first optical emitter such that light emission from the first optical emitter is pulsed. The first optical emitter emits light at a first wavelength and towards a chemical indicator. The method further includes detecting the chemical indicator’s first optical response to the light at the first wavelength. A second current level is applied to the second optical emitter such that light emission from the second optical emitter is pulsed. The second optical emitter emits light at a second wavelength and towards the chemical indicator, and the second current level is greater than the first current level. The method further includes detecting the chemical indicator’s second optical response to the light at the second wavelength. The method further includes estimating an analyte concentration based, at least in part, on the first optical response and the second optical response.

In Example 31, the method of Example 30, wherein the first optical response and the second optical response are detected by a photodiode.

In Example 32, the method of Example 30, further comprising: adjusting a gain of an optical detector in response to whether the first optical emitter or the second optical emitter is being pulsed.

In Example 33, the method of Example 30, wherein each pulse lasts 5–120 microseconds, wherein the first optical emitter is pulsed 5–100 times before the second optical emitter is pulsed 5–100 times.

In Example 34, the method of Example 30, wherein the analyte concentration is further based, at least in part, on an estimated temperature of the chemical indicator.

In Example 35, the method of Example 30, wherein the first wavelength is 510–570 nm, wherein the second wavelength is 610–740 nm.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration of a chemical sensing system, in accordance with certain instances of the present disclosure.

FIG. 2 shows an implantable medical device with a chemical sensor, in accordance with certain instances of the present disclosure.

FIG. 3 shows an exploded view of the implantable medical device of FIG. 2, in accordance with certain instances of the present disclosure.

FIG. 4 shows a side view of a portion of the implantable medical device of FIGS. 2 and 3, in accordance with certain instances of the present disclosure.

FIG. 5 shows a block diagram of a method, in accordance with certain instances of the present disclosure.

FIG. 6 shows a block diagram of circuitry of the implantable medical device of FIGS. 1-4, in accordance with certain instances of the present disclosure.

While the disclosed subject matter is amenable to various modifications and alternative forms, specific instances have been shown by way of example in the drawings and are described in detail below. The intention, however, is not to limit the disclosed subject matter to the particular instances described. On the contrary, the disclosed subject matter is intended to cover all modifications, equivalents, and alternatives falling within the scope of the disclosed subject matter as defined by the appended claims.

### DETAILED DESCRIPTION

Physiological parameters such as concentrations of certain analytes (e.g., levels of potassium, sodium, creatinine, calcium, blood urea nitrogen (BUN), and other analytes) can be measured and monitored to evaluate various physical conditions and performance such as a person’s kidney and/or cardiac conditions and performance.

Typically, measuring a person’s analyte concentrations requires drawing multiple blood samples from a patient at a clinic and then processing the blood samples at a laboratory. One approach for measuring analyte concentrations that does not require period blood draws, etc., is to use an implantable chemical sensor. An implantable chemical sensor can use electro-optical components such as light emitters and light detectors to sense one or more optical properties of the chemical sensor. Optical properties can used to estimate analyte concentrations.

Certain instances of the present disclosure are directed to approaches for detecting optical properties of chemical sensors.

### CHEMICAL SENSING SYSTEM

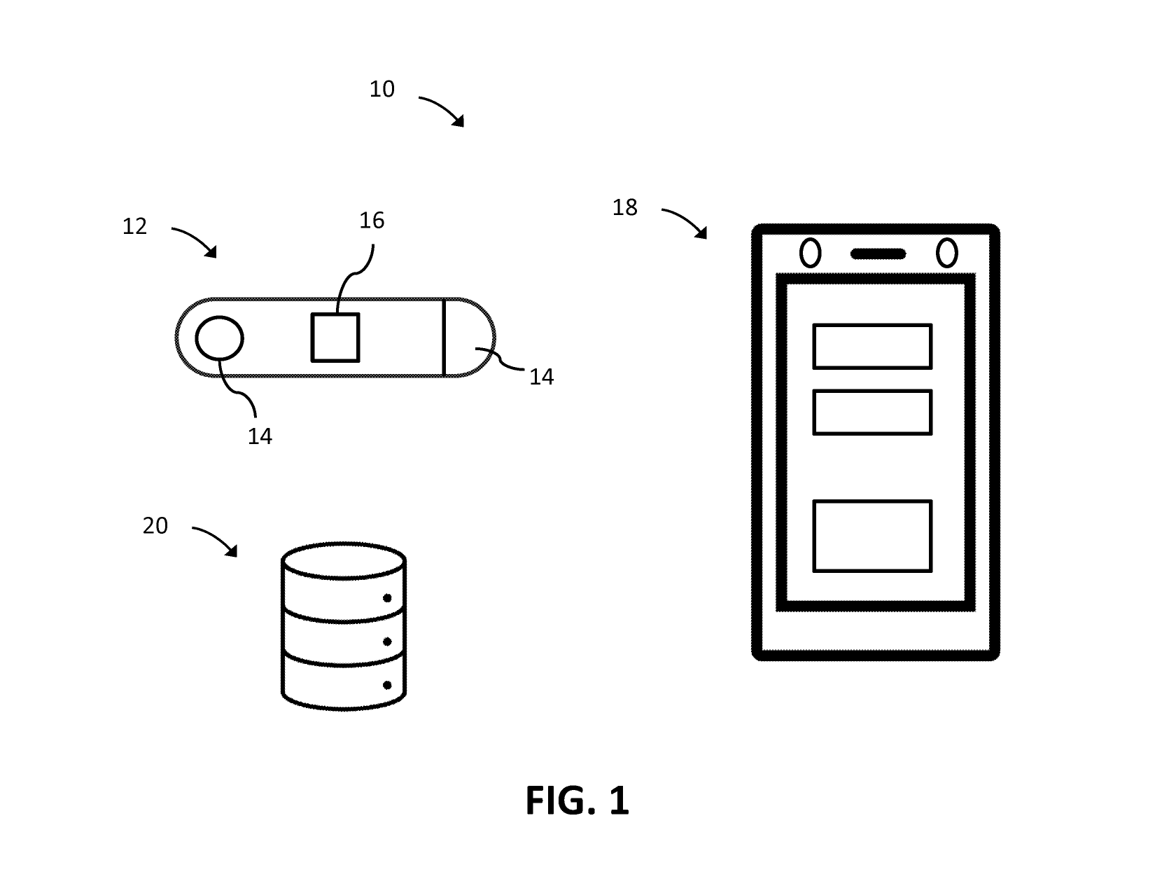

FIG. 1 shows a chemical sensing system 10 (hereinafter “the system 10” for brevity) with schematic representations of components that can be used to sense, measure, and monitor physiological parameters. In particular, components of the system 10 can ultimately be used to estimate analyte concentrations and pH levels using an implantable medical device.

The system 10 includes an implantable medical device 12, which is shown as including one or more electrodes 14 and a chemical sensor assembly 16. The electrodes 14 can comprise a conductive material and be configured to sense cardiac activation signals. Cardiac activation signals can be used to generate electrocardiogram (ECG) data. In some instances, the implantable medical device 12 does not include electrodes.

The chemical sensor assembly 16 can include a sensing element with a polymeric matrix permeable to analytes such as potassium, sodium, and/or creatinine and the like. The sensing element can include an interior volume with various chemical indicators (e.g., beads for detecting an ion concentration of a bodily fluid). Analytes can diffuse through an outer barrier layer and onto and/or into the chemical indicators where the analytes can bind with ion selective sensors to produce an optical response (e.g., a change in optical properties such as a change in concentration, a fluorimetric response, a colorimetric response). The optical response can be monitored and used to estimate analyte levels (e.g., analyte concentrations).

The system 10 can also include a computing device 18 such as a mobile computing device (e.g., a smart phone, a tablet, and the like) and/or a computing system 20 (e.g., a server). Estimated analyte levels can be used by a computing device 18 to monitor and evaluate a person’s kidney and/or cardiac performance among other functions. In certain instances, the implantable medical device 12 itself is programmed to estimate analyte levels based on optical properties of the chemical sensor. Additionally or alternatively, the computing device 18 and/or computing system 20 is programmed to estimate analyte levels, etc. The device 18 and/or the computing system 20 can communicate (e.g., wirelessly) with the implantable medical device 12 and each other.

### IMPLANTABLE MEDICAL DEVICE

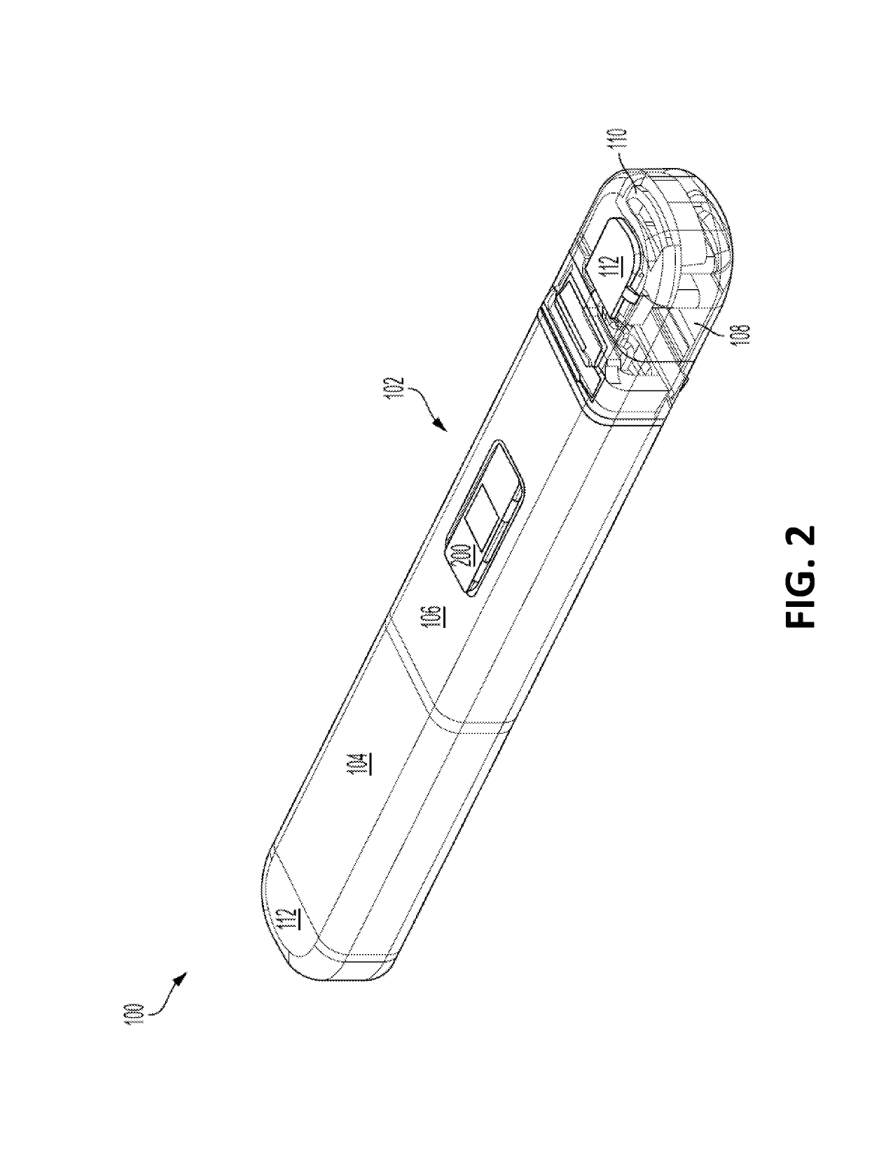

FIG. 2 shows an implantable medical device (IMD) 100 that includes a body 102 with various sections such as a battery module 104 (e.g., a section that houses a battery), an electronics housing 106 (e.g., a section that is hollow and houses various electronics such as a circuit board, integrated circuitry such as controllers and processors, and the like), a header 108 (e.g., a section that houses components such as an antenna 110), and electrodes 112 at opposite ends of the body 102. In certain instances, the IMD 100 is header-less.

The battery module 104 can include one or more electrochemical cells (e.g., rechargeable and/or single-use cells) disposed therein to provide power for the IMD 100. The electrochemical cell(s) can comprise a rechargeable lithium-based cell such as a lithium-manganese dioxide (Li anode/MnO₂ cathode) battery, however, other lithium battery chemistries are also contemplated herein — including, but are not limited to, CFx, SVO, hybrid CFx/Mn02, hybrid CFx/SVO, and the like.

The electrodes 112 comprise a conductive material and are arranged to sense cardiac activation signals. The sensed cardiac activation signals can be used to generate ECG data.

The IMD 100 also includes a chemical sensor assembly 200 (hereinafter “the chemical sensor 200” for brevity). The chemical sensor 200 can include one or more chemical indicators that are in communication (e.g., indirect communication) with a person’s blood. For example, the indicators may be exposed to interstitial fluid, which is in communication with blood. As described further herein, the chemical indicators can change optical properties as analyte levels change. The optical properties of the chemical indicator can be sensed and ultimately used to estimate analyte levels. Estimating an analyte level using the chemical sensor 200 can include sensing one or more optical properties of the chemical sensor 200 and estimating an analyte level based on the optical property. In certain instances, estimating an analyte level occurs periodically (e.g., every 30 minutes, once an hour) or on demand (e.g., when a patient or physician initiates the comparison). Although the chemical sensors may react in real-time (e.g., the chemical indicators change optical properties in real-time as analyte levels change in real-time), transmission of or estimating an analyte level less often can save computing and battery resources and may be preferable because analyte levels may not change drastically minute-by-minute.

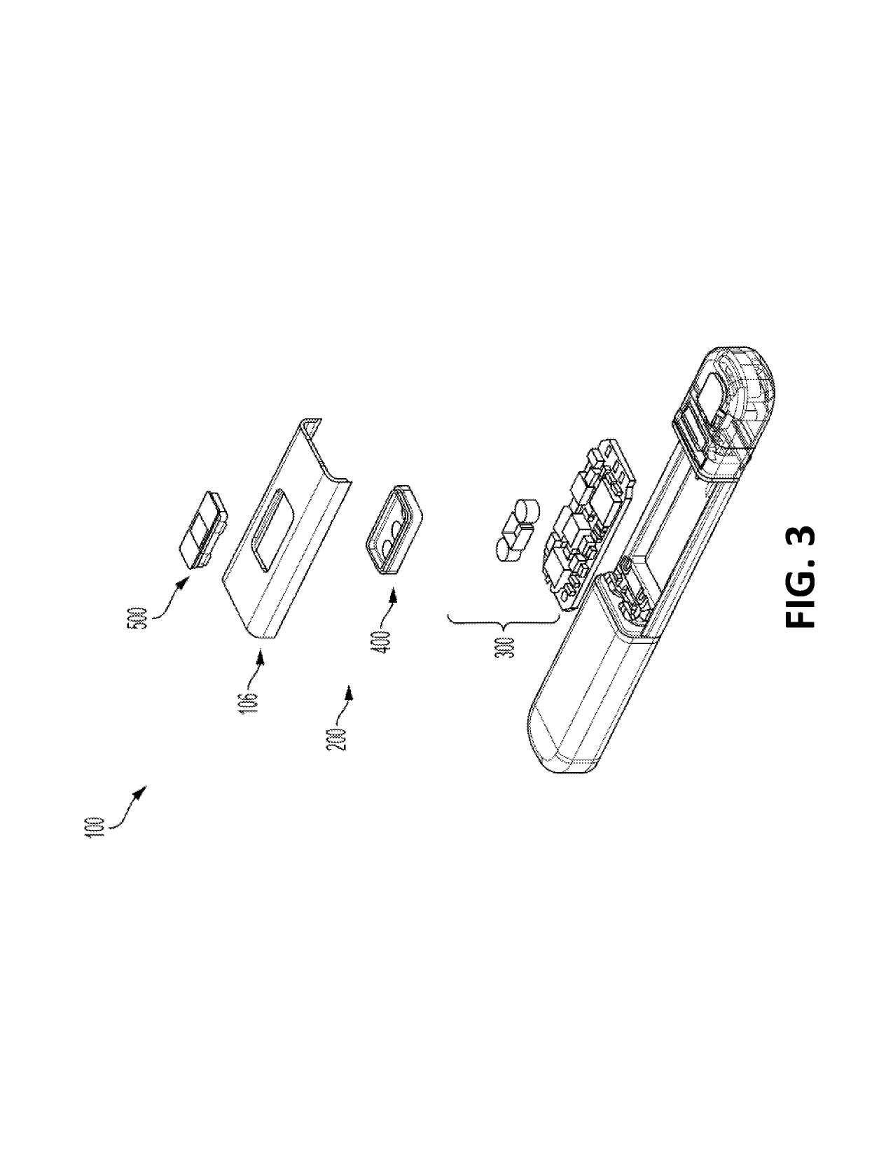

FIG. 3 shows an exploded view of the IMD 100 and, in particular, components of the chemical sensor 200. The chemical sensor 200 is described herein as including various subassemblies. The subassemblies include an opto-electronic assembly 300, an optical feedthrough 400 (hereinafter “the feedthrough 400” for brevity), and a chemical sensor cassette 500 (hereinafter “the cassette 500” for brevity).

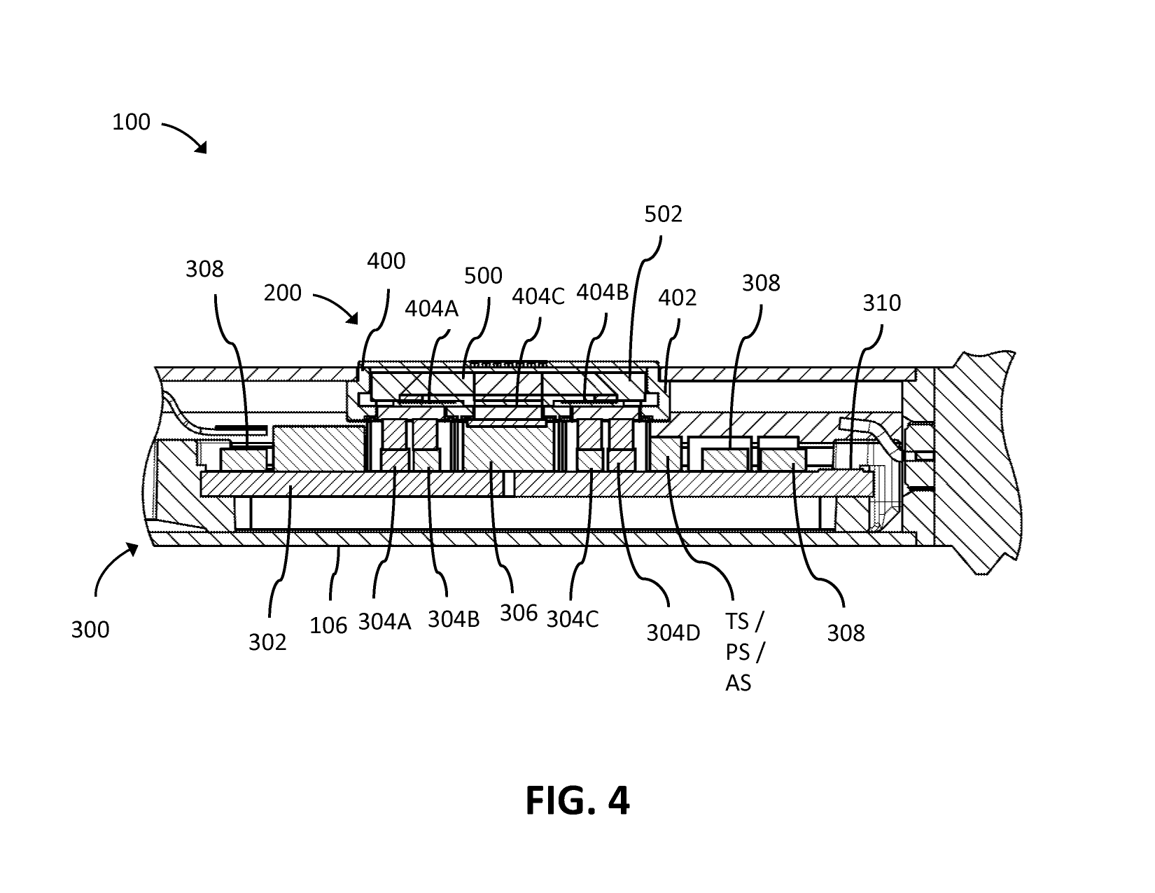

FIG. 4 shows a side view of a portion of the IMD 100 and the chemical sensor 200. As shown in FIG. 4, the opto-electronic assembly 300 portion of the chemical sensor 200 includes a circuit board 302, a first optical emitter 304A, a second optical emitter 304B, a third optical emitter 304C, and a fourth optical emitter 304D that are coupled to the circuit board 302. The circuit board 302 can include a wide variety of substrates with metallic conductors. For example, the circuit board 302 can include a ceramic substrate, a silicon wafer substrate, or other types of substrates with metallic conductors.

One or more surfaces (e.g., an upper surface to which the optical emitters are coupled to) of the circuit board 302 can include a layer or mask that is opaque to reduce reflection. For example, the solder mask used for various electrical connections can be made from an opaque material. The mask layer or mask can include carbon black and/or various pigments or components to render the layer or mask opaque. Additionally or alternatively, the circuit board 302 can be made using a resin that is opaque to reduce reflection. For example, instead of the circuit board 302 being a traditional “green board,” the circuit board 302 can be made of an opaque material such as a black resin.

In certain instances, the optical emitters 304A–D are solid state light sources such as light emitting diodes or laser diodes (e.g., GaAs, GaAlAs, GaAlAsP, GaAIP, GaAsp, Gap, GaN, InGaAIP, InGaN, ZnSe, or SiC light emitting diodes or laser diodes) that emit light that can excite a chemical indicator in the chemical sensor 200. In some instances, each optical emitter 304A–D is configured to emit light at a different wavelength (or band of wavelengths). In some instances, the optical emitters 304A–D are LEDs with band-limiting filters (e.g., high pass, low bass, band pass, band stop filters), and the filters can be interference-type or absorption-type filters.

Regardless of which respective specific wavelength (or band of wavelengths) each optical emitter is designed to emit, a given wavelength of emitted light can be at or near a wavelength of maximum absorption for at least one chemical indicator for a time sufficient to emit a return signal. However, it will be understood that, the wavelength of maximum absorption reflection may vary as a function of analyte level. Although the opto-electronic assembly 300 is shown has having four optical emitters, fewer or additional optical emitters can be used.

The opto-electronic assembly 300 also includes an optical detector 306 (e.g., a photodiode, charge-coupled device (CCD), junction field effect transistor (JFET) type optical sensor, or a complementary metal-oxide semiconductor (CMOS) type optical sensor) coupled to the circuit board 302. In certain instances, the optical detector 306 is the only optical detector of the opto-electronic assembly 300, but in other instances, multiple optical detectors can be used. The optical detector(s) 306 can also include one or more bandpass filters and/or focusing optics. For example, each optical detector can include one or more photodiode detectors, each with an optical bandpass filter tuned to a specific wavelength range.

Additional components can be coupled to the circuit board 302. These components include circuitry such as integrated circuits 308 (e.g., application specific integrated circuits (ASICs), field-programmable gate array (FPGAs), and the like) that can be programmed or controlled to carry out various functions described herein. For example, one integrated circuit 308 can include a controller (e.g., circuitry that includes a processor such as a microprocessor and memory that stores instructions for carrying out various functions), another integrated circuit 308 can include circuitry for driving the optical emitters (e.g., by applying current to the optical emitters to generate light), another integrated circuit 308 can include circuity for switching between the optical emitters (e.g., a switch such as a metal-oxide-semiconductor field-effect transistor (MOSFET) switch), and another integrated circuit 308 can be used to adjust gain of the optical detector 306.

Signals from the optical detector(s) 306 can be communicated to the circuitry (e.g., one of the integrated circuits 308 with microprocessor) for signal processing and analysis. For example, signals from the optical detector(s) 306 can be communicated via traces in the circuit board 302. The circuitry can perform various operations described further herein, including but not limited to detecting magnitudes of signal intensity, filtering operations, averaging signals, estimating analyte levels based on the signals, and the like. Outputs such as estimated analyte levels can be wirelessly communicated from the IMD 100 to a device and/or system external to the patient.

As shown in FIG. 4, the first optical emitter 304A and the second optical emitter 304B can be positioned on the one of the optical detector 306, and the third optical emitter 304C and the fourth optical emitter 304D can be positioned on an opposite side of the optical detector 306. Other arrangements can be used such as positioning all of the optical emitters on one side of the optical detector 306. Further, as shown in FIG. 4, each of the optical emitters 304A–D and the optical detector 306 can be directly coupled to a single side or surface 310 (e.g., a major surface) of the circuit board 302. However, the optical emitters 304A–D and the optical detector 306 could be coupled to separate circuit boards or separate surfaces.

As shown in FIG. 4, the opto-electronic assembly 300 is coupled to the electronics housing 106 and is positioned between the electronics housing 106 and the feedthrough 400. The feedthrough 400 includes a housing 402 with a bottom portion and a side wall portion surrounding a periphery of the bottom portion to create a well. The feedthrough 400 includes a first window 404A, a second window, 404B, and a third window 404C formed through the bottom portion of the feedthrough 400. Additional or fewer windows can be used. In the example of FIG. 4, the first window 404A is arranged above (e.g., immediately above) the first emitter 304A and the second emitter 304B such that emitted light passes through the first window 404A. The second window 404B is arranged above (e.g., immediately above) the third emitter 304C and the fourth emitter 304D such that emitted light passes through the second window 404B. The third window 404C is arranged above (e.g., immediately above) the optical detector 306 such that the optical detector 306 can sense optical properties of chemical indicators in the cassette 500.

When the IMD 100 is assembled, the cassette 500 is positioned within the feedthrough 400 (e.g., in the well of the feedthrough 400). The cassette 500 includes one or more chemical indicators 502 (shown in FIG. 3). The chemical indicators 502 can comprise a material that changes optical properties (e.g., color) with changes in concentration (or levels) of a given analyte — and such optical properties can be sensed by the optical detector 306. In certain instances, the color of the chemical indicator 502 comprises the sum of the absorption, transmission, reflectance, and fluorescence properties of the chemical indicator material.

The circuitry of the chemical sensor 200 of the IMD 100 can be used to carry out various functions relating to exciting and sensing the chemical indicators 502. These functions include managing and balancing power usage and signal-to-noise ratios (SNR). In certain instances, these functions are carried out by multiple separate integrated circuits 308. For example, one of the integrated circuits 308 can comprise a controller that controls the other integrated circuits 308.

The circuitry can be configured to apply a first current level to the first optical emitter 304A. For example, the current can be generated by one of the integrated circuits 308. In some instances, the current is applied to the first optical emitter 304A in pulses. For example, in some instances, each pulse of current can last 5–120 microseconds (e.g., 10–100, 20–90, 30–80 microseconds), and the number of pulses can be a predetermined number such as 5–100 individual pulses (e.g., 10–50, 20–40 pulses).

In certain instances, the circuitry applies the pulsed current a set number of times to the first optical emitter 304A before current is applied to another one of the optical emitters. As such, at any given point in time, only one optical emitter may be emitting light. After the pulsed current is applied to the first optical emitter 304A, the circuitry can be configured to apply a second current level to another optical emitter (e.g., the second optical emitter 304, the third optical emitter 304C, or the fourth optical emitter 304D). The current can be pulsed and repeated similarly to that described above for the first optical emitter 304A.

Because the wavelength of light emitted from the second optical emitter 304B is different than the wavelength of light emitted from the first optical emitter 304A, the level of current applied to respective optical emitters may be different to optimize power usage. For example, if the first optical emitter 304A emits light at a wavelength that is smaller than the second optical emitter 304A, then the level or amount of current applied to the first optical emitter 304A can be less than the level or amount of current applied to the second optical emitter 304B. As one specific example, an optical emitter that is configured to emit green light (e.g., wavelength of 510–570 nm) may require less current than an optical emitter configured to emit red light (e.g., wavelength of 610–740 nm). As such, because optical emitters that produce green light may be more efficient than optical emitters that produce red light, less current may be applied to “green” optical emitters compared to the current applied to “red” optical emitters. However, it is appreciated that the relationship of optical power per unit current may vary due to fabrication technique and design of a given optical emitter and an applied current for each optical emitter can be determined during calibration.

The circuitry can be configured to continue to cycle through the optical emitters 304A–D such that pulsed current is applied to each optical emitter to produce pulsed light a set number of times at set pulse lengths. In some instances, current is pulsed one-by-one to each of the optical emitters 304A–D in a cycle. After one cycle, the circuitry can be configured to pause for a predetermined period of time (e.g., 1–10 seconds) before cycling through the optical emitters again one-by-one. The level of current applied to each optical emitter can be different than the other optical emitters, as noted above.

In certain instances, the circuitry can be configured to adjust a gain of the optical detector 306 depending on which optical emitter is emitting light. For example, the circuitry can utilize a different level of gain of the optical detector 306 for each optical emitter 304A–D.

By varying the level of current applied to the optical emitters 304A–D and/or the level of gain of the optical detector 306, the circuitry can manage the power consumption of the chemical sensor 200. In the event that the output signal of the optical detector 306 contains too much noise (e.g., as determined by an SNR threshold or some other threshold), the circuitry can adjust (e.g., by increasing) the level of current and/or the level of gain to amplify or boost the signal ultimately detected by the optical detector 306.

Other techniques can be used to reduce the amount of noise in the signal detected by the optical detector 306.

One technique includes filtering out background light (e.g., filtering via background level subtraction). Some optical detectors can sense a wide range of frequencies (e.g., visible frequency range, infrared frequency range). The optical input of optical detector(s) can be filtered (e.g., bandstop filtered, bandpass filtered, low-pass filtered, high-pass filtered) to attenuate certain frequencies. For example, an optical filter can be positioned between the chemical indicators and the optical detector(s) to filter or attenuate wavelengths that are likely to be background light (e.g., sunlight that passes through the patient’s body and towards the optical detector(s)). Another approach for filtering out background light is to compare the detected optical signal when the optical emitter(s) are turn on with the detected optical signal when the optical emitter(s) are turned off to determine the content of the detected optical signal with and without the optical emitter(s).

Another technique includes applying signal averaging techniques to reduce noise in the output signals of the optical detector(s). Signal averaging can be used to reduce non-correlated noise (e.g., noise unrelated to the light emitted by the optical emitters). Examples of non-correlated noise that can be reduced using signal averaging include shunt noise, thermal noise. Digital or analog filters such as low pass filters, weighted average filters, finite impulse response (FIR) filters can be used to apply signal averaging to reduce non-correlated noise.

Additional techniques can be used to increase the accuracy of the estimations of analyte concentrations.

One technique includes applying temperature-based corrections to the optical properties (e.g., color) of the chemical indicators sensed by the optical emitter(s). Temperature can affect how chemical indicators absorb the light emitted by the optical emitters. As such, the estimated analyte concentrations can be based, at least in part, on an estimate temperature of the chemical indicator(s). To estimate temperature, the IMD 100 can include a temperature sensor (labeled “TS” in FIG. 4). The temperature sensor TS can be part of the circuitry coupled to the circuit board 302. For example, the temperature sensor TS could be part of an integrated circuit. As another example, the optical detector 306 itself may be capable of estimating temperature. Regardless of which component estimates the temperature, the temperature sensor can be located close to the chemical indicators to increase the accuracy of the estimated temperature. The estimated temperature can be used in connection with a calibration curve or table to make the temperature-based corrections to the sensed optical properties of the chemical indicators. In some instances, if the estimated temperature is within a “normal” temperature range (e.g., 36–38 °C), no temperature-based correction is applied. However, if the estimated temperature is outside of the range, then a calibration curve or table is used to make a temperature-based correction.

Another technique includes applying pressure-based corrections to the optical properties (e.g., color) of the chemical indicators sensed by the optical emitter(s). In some situations, the IMD 100 can be compressed which can affect how analytes flow or otherwise interact with the chemical indicators. The IMD 100 can subjected to pressure, for example, if the patient is sleeping on their stomach and part of their body weight compresses the IMD 100. As such, the estimated analyte concentrations can be based, at least in part, on an estimate pressure. To estimate temperature, the IMD 100 can include a pressure sensor (labeled “PS” in FIG. 4). The pressure sensor PS can be part of the circuitry coupled to the circuit board 302. For example, the pressure sensor PS could be part of an integrated circuit. The estimated pressure can be used in connection with a calibration curve or table to make the pressure-based corrections to the sensed optical properties of the chemical indicators.

In certain instances, the IMD 100 can include a posture sensor such as an acceleration sensor (labeled “AS” in FIG. 4). The posture sensor can be used to detect patient activity and/or posture, which can be used to estimate whether a patient is in an active state or sleep state. In certain instances, if the patient is estimated to be in an active state, then (to save power) the pressure sensor PS is not interrogated and instead is only interrogated when the patient is estimated to be sleeping.

Another technique includes normalizing the output signal of the optical detector relative to the isosbestic wavelength (or isosbestic point) of the chemical indicator. For example, if the isosbestic wavelength of a given chemical indicator is 540 nm, then the wavelength of the chemical indicator(s) detected by the optical detector 306 can be divided by 540. The isosbestic wavelength provides a reference point that can be used to compensate for optical path variations and that is independent of the saturation state of the chemical indicator(s). The normalized output signal (e.g., the ratio of the detected wavelength divided by the isosbestic wavelength) can be used in connection with a calibration curve or table to make the corrections to the sensed optical properties of the chemical indicators.

The various calibration curves or tables described herein can be generated during manufacture and testing of the IMD 100 and/or in-the-field. During manufacture, the chemical indicator(s) can be exposed to known analyte concentrations while parameters such as temperature, pressure, optical properties, etc., are varied and correlated to the known analyte concentrations. Once the IMD 100 is implanted, the IMD 100 can be calibrated periodically. This can involve estimating analyte concentrations by the IMD 100 and also having the patient have their blood drawn and analyte concentrations measured by a laboratory. The estimated concentrations and measure concentrations can be determined and used to recalibrate the IMD 100.

### CHEMICAL INDICATOR

In certain instances, the chemical indicator 502 (FIG. 4) is formed of a lipophilic indicator dye (e.g., a lipophilic fluorescent indicator dye or a lipophilic colorimetric indicator dye). Lipophilic indicator dyes can include, but are not limited to, ion selective sensors such as ionophores or fluorophores. In certain instances, ionophores can include sodium-specific ionophores, potassium-specific ionophores, calcium-specific ionophores, magnesium-specific ionophores, and lithium-specific ionophores. In certain instances, fluorophores can include lithium-specific fluorophores, sodium-specific fluorophores, and potassium-specific fluorophores.

Compositions of the chemical indicator 502 can include components (or response elements) that are configured for a colorimetric response, a photoluminescent response, or another optical sensing modality. For example, the chemical indicator 502 can include an element that changes color based on binding with or otherwise complexing with a specific chemical analyte. As one specific example, creatinine reacts with a molecule which changes pH and color on the indicator. In some instances, the chemical indicator 502 can include a complexing moiety and a colorimetric moiety. Those moieties can be a part of a single chemical compound (e.g., a non-carrier-based system) or can be separated on two or more different chemical compounds (e.g., a carrier-based system). The colorimetric moiety can exhibit differential light absorbance on binding of the complexing moiety to an analyte.

Some of the chemical indicators 502 may not require a separate compound to both complex an analyte of interest and produce an optical response. By way of example, in some instances, the response element can include a non-carrier optical moiety or material wherein selective complexation with the analyte of interest directly produces either a colorimetric or fluorescent response. As an example, a fluoroionophore can be used and is a compound including both a fluorescent moiety and an ion complexing moiety. As merely one example, (6,7-[2.2.2]-cryptando-3-[2″-(5″-carboethoxy)thiophenyl]coumarin, a potassium ion selective fluoroionophore, can be used (and in some cases covalently attached to polymeric matrix or membrane) to produce a fluorescence-based K+ non-carrier response element. An exemplary class of fluoroionophores are the coumarocryptands. Coumarocryptands can include lithium specific fluoroionophores, sodium specific fluoroionophores, and potassium specific fluoroionophores. For example, lithium specific fluoroionophores can include (6,7-[2.1.1]-cryptando-3-[2″-(5″-carboethoxy)furyl]coumarin. Sodium specific fluoroionophores can include (6,7-[2.2.1]-cryptando-3-[2″-(5″-carboethoxy)furyl]coumarin. Potassium specific fluoroionophores can include (6,7-[2.2.2]-cryptando-3-[2″-(5″-carboethoxy)furyl]coumarin and (6,7-[2.2.2]-cryptando-3-[2″-(5″-carboethoxy)thiophenyl]coumarin.

### METHODS

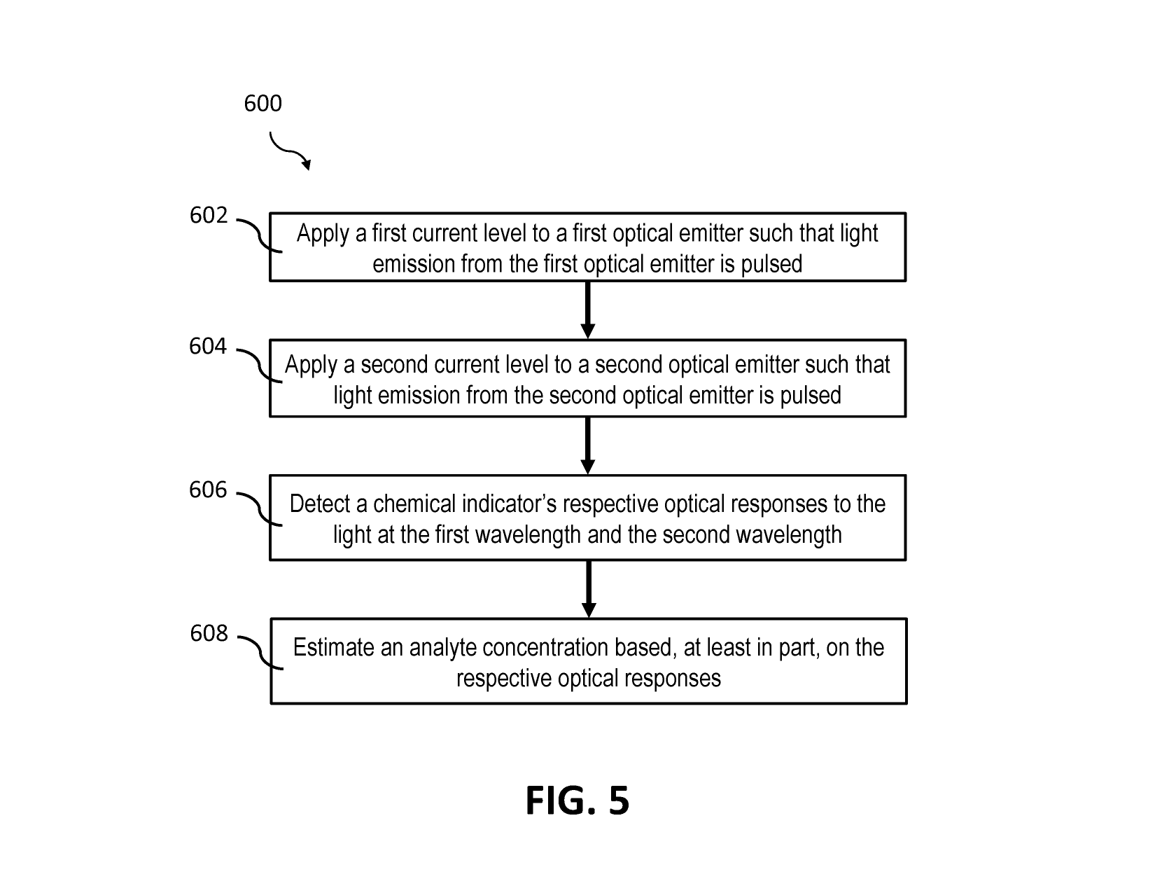

FIG. 5 outlines a method 600 that can be used in connection with one or more components of the system 10 of FIG. 1 and the IMD 100 of FIGS. 2-4.

The method 600 includes applying a first current level to a first optical emitter such that light emission from the first optical emitter is pulsed (block 602 in FIG. 5). The first optical emitter is configured to emit light at a first wavelength and towards a chemical indicator. The method 600 further includes applying a second current level to a second optical emitter such that light emission from the second optical emitter is pulsed (block 604 in FIG. 5). The second optical emitter emits light at a second wavelength (different than the first wavelength) and towards the chemical indicator. The second current level is different than the first current level. Additional optical emitters and respective wavelengths can be used to interrogate the chemical indicator(s).

The chemical indicator’s respective optical responses to the light at the first wavelength and the second wavelength are detected (block 606 in FIG. 5) by an optical detector as described herein. The respective optical responses are both used to estimate an analyte concentration (block 608 in FIG. 5).

### CIRCUITRY

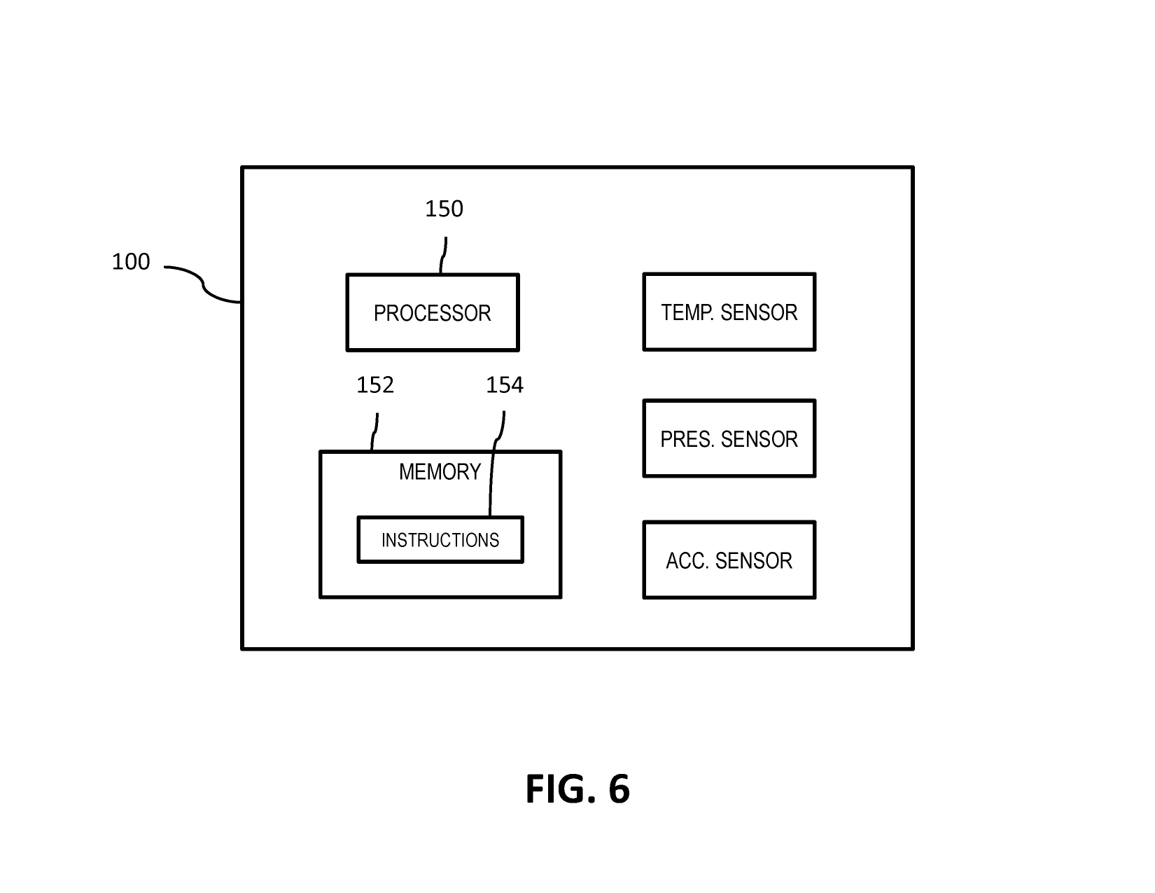

FIG. 6 shows a block diagram of certain circuitry of the IMD 100. The circuitry includes a processor 150 such as a microprocessor. The circuitry also includes memory 152 and instructions 154. The instructions 154 may be configured to be executed by the processor 150 and, upon execution, to cause the processor 150 to perform certain processes and functions described herein. The processor 150, memory 152, and instructions 154 can be part of a controller such as a controller used by an application specific integrated circuit (ASIC), field-programmable gate array (FPGA), and/or the like. Such devices can be used to carry out the functions and steps described herein.

In certain instances, the memory 152 includes computer-readable media in the form of volatile and/or nonvolatile memory and may be removable, nonremovable, or a combination thereof. Media examples include random access memory (RAM), read only memory (ROM), electronically erasable programmable read only memory (EEPROM), flash memory, and/or any other medium that can be used to store information and can be accessed by a computing device such as the processor 150. In instances, the memory stores the computer-executable instructions 154 for causing the processor 150 to implement aspects of instances of components discussed herein and/or to perform aspects of instances of methods and procedures discussed herein. The memory 152 can comprise a non-transitory computer readable medium storing the computer-executable instructions 154.

The computer-executable instructions 154 may include, for example, computer code, machine-useable instructions, and the like such as, for example, program components capable of being executed by one or more processors associated with the computing device 150. Program components may be programmed using any number of different programming environments, including various languages, development kits, frameworks, and/or the like. Some or all of the functionality contemplated herein may also, or alternatively, be implemented in hardware and/or firmware.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

WE CLAIM:

1. An apparatus comprising:
an opto-electronic assembly that includes:
a substrate,
a first optical emitter coupled to the substrate and configured to emit light at a first wavelength, and
a second optical emitter coupled to the substrate and configured to emit light at a second wavelength different than the first wavelength; and
circuitry configured to:
apply a first current level to the first optical emitter, and
apply a second current level to the second optical emitter, wherein the second current level is greater than the first current level.

2. The apparatus of claim 1, wherein the circuitry is further configured to:
apply the first current level to the first optical emitter such that light emission from the first optical emitter is pulsed, and
apply the second current level to the second optical emitter such that light emission from the second optical emitter is pulsed.

3. The apparatus of claim 1, wherein the circuitry is further configured to:
apply the first current level to the first optical emitter such that light emission from the first optical emitter is pulsed a first set number of times before the second current level is applied to the second optical emitter such that light emission from the first optical emitter is pulsed a second set number of times.

4. The apparatus of claim 3, wherein the first set number is 5–100, wherein the second set number is 5–100.

5. The apparatus of claim 3, wherein each pulse lasts 5–120 microseconds.

6. The apparatus of claim 1, wherein the opto-electronic assembly further includes an optical detector coupled to the substrate.

7. The apparatus of claim 6, wherein the circuitry is further configured to adjust a gain of the optical detector in response to whether the first optical emitter or the second optical emitter is being pulsed.

8. The apparatus of claim 6, wherein the circuitry is further configured to apply a signal averaging technique to reduce noise in a signal generated by the optical detector.

9. The apparatus of claim 1, wherein the apparatus is a medical device, wherein the medical device includes a chemical indicator arranged to reflect light from the first optical emitter and the second optical emitter, wherein the medical device is programmed to estimate an analyte concentration based, at least in part, on an optical property of the chemical indicator.

10. The apparatus of claim 9, wherein the analyte concentration is further based, at least in part, on an estimated temperature of the chemical indicator.

11. The apparatus of claim 9, wherein the analyte concentration is further based, at least in part, on an estimated pressure.

12. The apparatus of claim 9, wherein the analyte concentration is further based, at least in part, on a sleep state or active state of a patient.

13. The apparatus of claim 1, wherein the first optical emitter and the second optical emitter are light emitting diodes.

14. The apparatus of claim 1, wherein the first wavelength is 510–570 nm, wherein the second wavelength is 610–740 nm.

15. A method for use with an implantable medical device with a substrate, a first optical emitter coupled to the substrate, and a second optical emitter coupled to the substrate, the method comprising:
applying a first current level to the first optical emitter such that light emission from the first optical emitter is pulsed, wherein the first optical emitter emits light at a first wavelength and towards a chemical indicator;
detecting the chemical indicator’s first optical response to the light at the first wavelength;
applying a second current level to the second optical emitter such that light emission from the second optical emitter is pulsed, wherein the second optical emitter emits light at a second wavelength and towards the chemical indicator, wherein the second current level is greater than the first current level;
detecting the chemical indicator’s second optical response to the light at the second wavelength; and
estimating an analyte concentration based, at least in part, on the first optical response and the second optical response.

16. The method of claim 15, wherein the first optical response and the second optical response are detected by a photodiode.

17. The method of claim 15, further comprising: adjusting a gain of an optical detector in response to whether the first optical emitter or the second optical emitter is being pulsed.

18. The method of claim 15, wherein each pulse lasts 5–120 microseconds, wherein the first optical emitter is pulsed 5–100 times before the second optical emitter is pulsed 5–100 times.

19. The method of claim 15, wherein the analyte concentration is further based, at least in part, on an estimated temperature of the chemical indicator.

20. The method of claim 15, wherein the first wavelength is 510–570 nm, wherein the second wavelength is 610–740 nm.
